(19) United States
(12) Patent Application Publication
(10) Pub. No.: US 2008/0026008 A1
(43) Pub. Date: Jan. 31, 2008

(54) Bacteriophage DNA vaccine vector

(76) Inventors: Clark Tibbs, Newark, OH (US); Dale Vander Putten, Hagerstown, MD (US)

Correspondence Address: Thomas A. O'Rourke;Bodner & O'Rourke, 425 Broadhollow Road, Melville, NY, 11747, US

(21) Appl. No.: 11/879,216

(22) Filed: Jul. 16, 2007

Related U.S. Application Data
(60) Provisional application No. 60/833,492, filed on Jul. 27, 2006.

(51) Int. Cl. A61K 39/145; A61K 39/12; A61P 31/12

(52) U.S. Cl. 424/209.1; 424/204.1

(57) ABSTRACT

Bacteriophages are used as a DNA delivery system for a vaccine against pathogens where glycosylation is critical to immunity.

This application claims priority on U.S. Application Ser. No. 60/833,492 filed on Jul. 27, 2006, the disclosures of which are incorporated herein by reference.

### FIELD OF THE INVENTION

The present invention relates to improvements in the preparation of vaccines for the prevention of viral based diseases and more particularly, vaccines for the treatment of influenza.

### BACKGROUND OF THE INVENTION

The rapid development and deployment of prophylactic and therapeutic vaccines remains a serious problem that is worsening with increased ease of global travel and emerging infectious diseases on the rise. The current technologies for developing and scaling vaccines were most appropriate for an earlier time when infections moved slowly around the globe and surveillance was capable of providing several months to years of warning of an impending outbreak of an existing or emerging pathogen. Several recent infectious diseases outbreaks provide evidence that this extended period of warning to emergence of an epidemic or pandemic is shortening and at an accelerated pace. Recent SARS and avian influenza outbreaks are prime examples of this phenomenon.

As the world has gotten smaller, diseases that were once relegated to certain parts of the world now can become worldwide because of the availability of air travel and widespread international trade. Because of the vast scope of international trade in the global economy, a number of diseases have spread far beyond their normal boundaries. In addition, many diseases are able to spread more quickly due to, for example, the large volume of air travel. Global warming has also been postulated as having an effect on the spread of disease as well.

Influenza is one such disease that has benefited from globalization. One strain of Avian flu, A(H5N1) virus has been known in certain areas of Asia for a number of years, but its spread had been fairly limited. More recently, outbreaks of the virus have been seen in widely dispersed areas such as India, Egypt and France that has left international health experts concerned and questioning why the disease is moving so rapidly after being relegated to one area for many years.

Influenza appears in seasonal epidemics caused primarily by new strains that can result from the spread of an existing flu virus to humans from another animal species. The avian influenza H5N1 that was first discovered in the 1990s was thought to be a prime candidate for causing an influenza pandemic. However, this virus has not so far mutated to spread easily between people.

Although there are vaccines available for influenza, these are often of limited value. Most influenza vaccines are trivalent in that they include purified and inactivated viruses of three discrete strains. These strains can vary each year as the trivalent influenza vaccine is formulated annually based on influenza strains projected to be prevalent.

Because it can take a significant period of time to develop the vaccine and test it, there is a high risk that the selected influenza strains are not particularly useful against a given years actual influenza strain. The influenza virus, like all RNA viruses, has a high mutation rate and therefore, over time, these mutations accumulate and eventually the virus evolves into a new strain.

Current methods for the development of vaccines against these emerging infections are too slow to provide in time protection when faced with a virus that mutates rapidly. There is a high risk that the virus can spread quickly before a vaccine can be developed. As a result, there is a need for a just in time manufacturing system to address this problem.

Current influenza vaccine production using the well established chicken egg technology, has great difficulty producing enough vaccine on time for the flu season even with a one year notice. In addition, the vaccines made in this manner are contraindicated for people with egg allergies. In the case of pandemic flu, there will not be a one year advance notice and obtaining purified starting material for vaccine deployment will take more time than is available to prevent broad dissemination of the disease.

It has been proposed that mammalian cell manufacture will have significant advantages over manufacturing in eggs. Although it is more easily scaled, mammalian manufacture does not attenuate the virus and can produce actual pathogenic virus rather than desired strain. More importantly, while larger batches of vaccine may be available earlier with mammalian cell manufacture, the overall gain in production volume (i.e doses) is not significant. The epidemiology of a pandemic is such that mammalian cell produced vaccine would not be available for the first wave of infection where millions may die; vaccine produced in either egg or mammalian cell culture would be available for a second wave of infection.

The only vaccine manufacturing technology that has the possibility of producing vaccine rapidly enough to protect against the first wave of a pandemic is to use a bacterial host such as E. Coli for expression of protective protein antigens. However the manufacture of such a vaccine in E. Coli is not straightforward. In the case of influenza, where glycosylation of the influenza proteins is critical to providing a protective immune response, E.Coli or other bacterially based manufacture would not be feasible since those systems do not glycosylate protein products. In addition, E. Coli and other bacteria do not serve as hosts for the influenza virus thus making whole, attenuated, or killed virus production impossible in that background. E. Coli and other bacteria can replicate DNA plasmids that contain viral sequences but recent research has shown that direct DNA administration does not produce a protective antibody response. E. Coli and other bacteria can serve as hosts for the production of bacterial viruses, bacteriophage (phage). Recent work has shown that phage can be engineered to express foreign protein sequences on their surface that can act as vaccines. However, as with other proteins expressed in bacteria, these proteins are not glycosylated and thus the constructs are ineffective against pathogens where glycosylation is important in immunity.

Recently, March and his collaborators have demonstrated that by placing the DNA sequence coding for a known protective antigen of hepatitis B into phage and injecting this recombinant phage into a laboratory animal, a protective response is obtained against hepatitis infection. (Vaccine (2004) April 16;22(13-14):1666-71 Genetic immunization against hepatitis B using whole bacteriophage lambda particles. March J B, Clark J R, Jepson C D. Vaccine (2004) June 23;22(19):2413-9 Bacteriophage lambda is a highly stable DNA vaccine delivery vehicle. Jepson C D, March J B.)

The target antigen is not expressed on the surface of the phage but rather the phage apparently serves as an effective transfection agent for the animal host cells thereby allowing them to produce the foreign protein. It is important to note that the Hep B vaccine is already produced in E. Coli as a protein subunit vaccine (no glycosylation required) and there is no time urgency in the manufacture of hep B vaccine which is already widely available while less susceptible to seasonal variation. Accordingly, March's use of phage to produce a vaccine where glycosylation and urgency of scalability is irrelevant is very different from the use described herein.

### OBJECTS OF THE INVENTION

It is an object of the invention to provide a vaccine for preventing viral infections.

It is also an object of the invention to provide a vaccine for RNA viral infections.

It is another object of the invention to provide a vaccine for influenza infections.

It is a further object of the invention to provide an improved method of manufacturing vaccines.

It is still a further object of the invention to provide a just in time method of manufacturing vaccines.

It is still another object of the invention to provide a method of manufacturing vaccines using a bacteriophage vector.

It is a further object of the invention to provide a method of manufacturing a vaccine using a bacteriophage vector produced in E. Coli.

### SUMMARY OF THE INVENTION

The present invention provides a new method of vaccine production based on early identification and genetic characterization allowing for generation of scalable vaccine production that is effective and rapid. The present invention is superior to existing and proposed technologies in that: 1) it only requires gene sequence information from the pathogen, 2) the vaccine is rapidly generated without purification and culture of the pathogen, 3) the vaccine is non-pathogenic so it is safe and requires no inactivation, attenuation, or subunit purification, 4) the vaccine is more rapidly scalable than competing technologies, especially where proper glycosylation is important in providing immunity. Using phage as a DNA delivery mechanism for a vaccine against pathogens where glycosylation is critical to immunity is novel and would be unexpected. Phage does not replicate in mammalian cells but the described system allows for host cell production and preserves critical post-translational modifications such as glycosylation that may be critical for development of a protective response.

This system is a plug and play component system where once a sequence is identified, the time to a clinical production lot is reduced from months or years to weeks. Direct experimentation has shown that H5 immunity in mice can be generated starting from the readily available gene sequence using a bacteriophage vector produced in E. Coli.

It has been found that pathogens such as emerging infections, bio-warfare agents, or pathogens evolving resistance to treatment where urgency is important and the role of glycosylation is known to be important or is unknown, can be treated with vaccines made by our integrated method of 1) identifying a likely or known protective gene 2) synthesis of the candidate gene 3) insertion of the gene into phage 4) production in bacteria of lots of the phage containing the candidate gene. We have demonstrated this principle using H5N1 influenza. In addition to incorporating the desired gene sequence of the target antigen into the phage DNA, additional modifications can be made to further improve the host immune response. Two such involve the addition of a mammalian signal sequence at the 5′-end of the DNA, and a second is the introduction of the DNA sequence that codes for the addition of a 3′-glycosylphosphatidylinositol anchor structure. The former assists in directing newly synthesized protein into a trafficking pathway within the cell that ensures exposure to the glycosylation machinery while the latter serves to immobilize the protein product on the surface of the cell, increasing local concentration and improving potential interaction with cells of the host immune system.

Essentially, using optimized production techniques (in terms of growth media, growth conditions, host E. coli strains and a wild type phage, the yield can be as high as 10*15 phage per liter of culture. A 100 Liter Bioreactor can produce 10*17 phage. At a dose of 10*10 phage, this would equate to 10 million doses; (based on animal weight factors as discussed below). The mechanism is believed to be resulting in the translation in these cells of the vector DNA sequence, followed by appropriate glycosylation of the resulting protein product, resulting in an immune response by the host. The mechanism meets the criterion of very rapidly creating a vaccine with only DNA sequence information. It is scalable in E. Coli, and the appropriate antigen/s will be appropriately glycosylated.

The inclusion of an IRET (Immune Response Enhancement Technology) approach is broadly applicable to the bacteriophage-DNA platform. In essence this involves addition of a DNA sequence to the 5′-end of the protective antigen. This sequence codes for a mammalian signal sequence and ensures direction of the synthesized protein into the secretory pathway and thence to the cell surface. In addition, a second sequence can be added at the 3′ end that codes for addition of a GPI anchor. The latter is a capability of all cells and serves to immobilize the target protein at the cell surface so that the entire amino acid sequence is exo-directed.

Using phage as a DNA delivery for a vaccine against pathogens where glycosylation is critical to immunity is unique and unexpected. The DNA sequence of a key antigen from the H5 strain of Avian influenza virus was synthesized and cloned into a plasmid for expansion. Excision with restriction endonuclease allowed insertion of the gene sequence into phage lambda using standard methods. The sequence of the inserted fragment was confirmed and recombinant phage prepared in an E. coli host. Phage were purified after lysis by centrifugation and used to immunize mice.

### BRIEF DESCRIPTION OF THE FIGURES

FIG. 1 is a representation showing anti-H5 reactivity over a period of 56 days.

FIG. 2 is graph showing the effect of dilution of the serum anti-H5 reactivity.

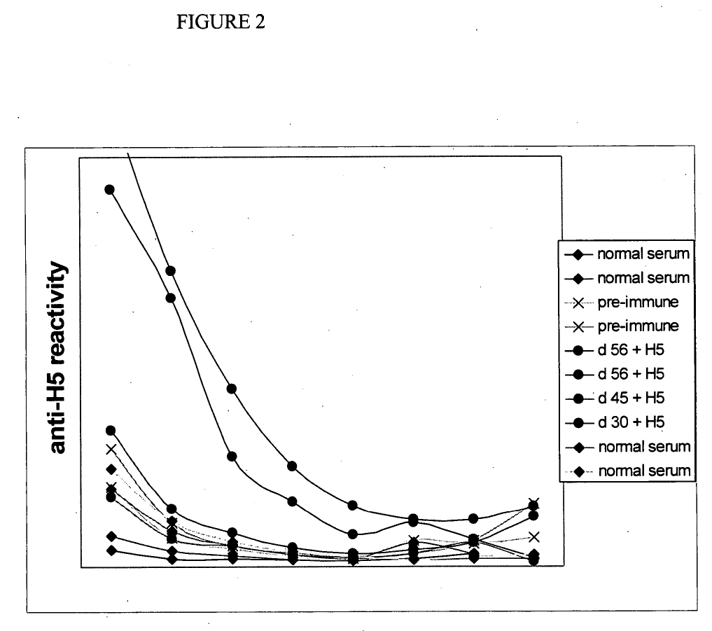

FIG. 3 shows the amounts of vaccine in normal saline without adjuvant injected on days 0, 15, and 45

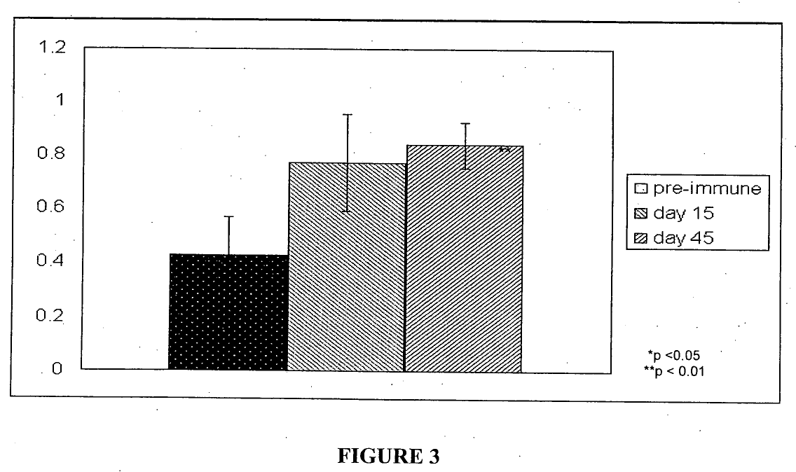

### DETAILED DESCRIPTION OF THE INVENTION

In the present invention, a virus vaccine is prepared. The DNA sequence of a virus for which a vaccine is desired is obtained. The complete gene for the virus is synthesized by standard methods and cloned into a plasmid. The gene fragment so produced is sequenced in both directions by established methods to confirm its identity.

The DNA sequence of interest is inserted into a bacteriophage by excision of the fragment and ligation into the phage using methods well known to those versed in the art. The DNA is packaged into phage heads and the resultant phage grown in E. Coli. Plaques (representing phage-infected bacterial cells) are picked and the isolated phage expanded in standard culture medium. After expansion, the plaques are extracted by lysis and the phage purified by centrifugation, then titered. The resultant phage can be administered to the patient.

The viruses that the present invention is particularly useful with are RNA viruses. These viruses typically belong to either Group III, Group IV or Group V of the Baltimore classification system. As such they possess ribonucleic acid (RNA) as their genetic material. The nucleic acid is typically a single stranded RNA (ssRNA), but can in some instances double stranded RNA (dsRNA).

Typical human pathogenic RNA viruses include SARS, influenza and hepatitis C. The present invention has particular applicability to viruses that rely on the expression of specific oligosaccharides for functions such as entry into host cells, correct proteolytic processing and protein trafficking. Examples of such viruses include but are not limited to Hendra, SARS, CoV Influenza and hepatitis C.

### EXAMPLE

Avian Influenza H5 sequence was obtained from Genbank (AY651330 A/bird/Thialand/3.1/2004 (H5N1) HA). (Li, K. S. et al., Genesis of a highly pathogenic and potentially pandemic H5N1 influenza virus in eastern Asia. Nature 430 (6996), 209-213 (2004)) The virus was characterized at Queen Mary Hospital in Hong Kong and reported in a manner typical of emerging pathogens.

The complete H5 gene was synthesized and the gene product cloned into a pJ4:G03267 plasmid. The fragment had the expected size by agarose gel electrophoresis, and was sequenced in both directions to confirm identity.

The H5 DNA was inserted into lambda bacteriophage (Uni-ZAP XR Vector Kit and Gigapack cloning kit from Stratagene) by EcoR I excision of the fragment and ligation into the phage.

The sequence of the inserted sequence was confirmed by PCR and DNA sequencing. The DNA was packaged into phage heads and grown in E.Coli on LB plates. Plaques were picked and expanded in LB broth, extracted by lysis, purified by centrifugation then titered.

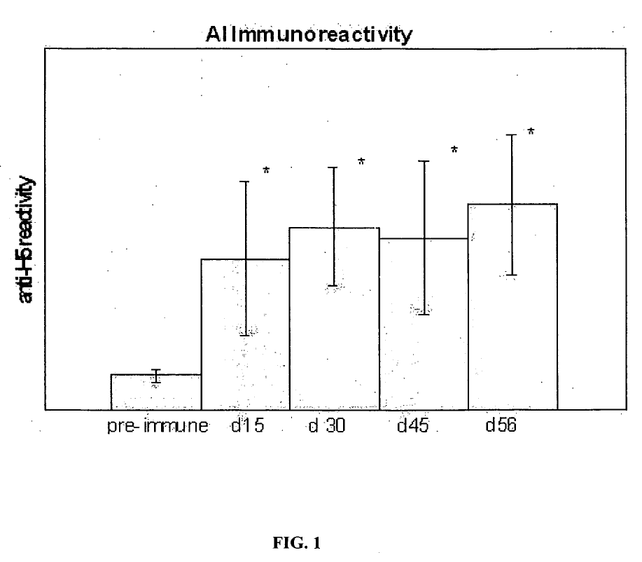

Mice were immunized intramuscularly with 10⁸ pfu of vaccine in normal saline without adjuvant on days 0, 15, and 45 and tested for antibody production on days 0, 15, 30, 45 and 56. See FIG. 1.

The antibody response in the mice was determined by the use of an IDEXX FlockChek avian influenza virus antibody ELISA kit that is licensed by the USDA for detecting avian influenza serum antibodies in chickens. The test was modified using KPL HRP conjugated goat anti-murine IgG+IgM(H+L) secondary antibody and the KBL SUREBLUE TMB kit for detection.

1. A method of treating a viral infection comprising providing a therapeutic amount of a vaccine created by a bacteriophage vector.

2. The method according to claim 1 wherein said bacteriophage vector is produced in E. Coli.

3. The method according to claim 2 wherein said viral infection is caused by an RNA virus.

4. The method according to claim 3 wherein said RNA virus is a Group III virus.

5. The method according to claim 3 wherein said RNA virus is a Group IV virus.

6. The method according to claim 3 wherein said RNA virus is a Group V virus.

7. The method according to claim 3 wherein said RNA virus is influenza.

8. The method according to claim 7 wherein said influenza virus is Avian Influenza.

9. The method according to claim 7 wherein the influenza virus is H5N1.

10. A vaccine effective for the protection of humans against a virus where said virus is one where glycosytatin of the virus proteins is critical to providing protective immunity comprising an immunologically effective amount of plaque formed by the insertion of the influenza DNA into a bacteriophage said bacteriophage being grown on E.Coli and a pharmaceutically acceptable carrier.

11. The vaccine according to claim 10 wherein said virus is an RNA virus.

12. The vaccine according to claim 11 wherein said virus is a Group III virus.

13. The vaccine according to claim 11 wherein said virus is a Group IV virus.

14. The vaccine according to claim 11 wherein said virus is a Group V virus.

15. The vaccine according to claim 11 wherein said RNA virus is influenza.

16. The vaccine according to claim 15 wherein said influenza is Avian Influenza.

17. The method according to claim 16 wherein the influenza virus is H5N1.

18. The vaccine according to claim 17 wherein said plague is formed by obtaining a DNA sequence of said virus, characterizing said virus, synthesizing the DNA by cloning into a plasmid inserting said DNA into a bacteriophage, packaging the DNA into phage heads and growing on E.Coli.

19. The vaccine according to claim 18 wherein a mammalian signal sequence is added at the 5′-end of the DNA.

20. The vaccine according to claim 18 wherein a DNA sequence that codes for the addition of a 3′-glycosylphosphatidylinositol anchor structure is introduced into the DNA.

21. A method of making a vaccine for treatment of a viral infection caused by a virus wherein glycosylation of the virus proteins is critical to providing protective immunity comprising obtaining DNA sequence of said virus, characterizing said virus, synthesizing the DNA by cloning onto a plasmid inserting said DNA into a bacteriophage, packaging said DNA into phageheads and growing on E.Coli.

22. The method according to claim 21 wherein a mammalian signal sequence is added at the 5′-end of the DNA.

23. The method according to claim 21 wherein a DNA sequence that codes for the addition of a 3′-glycosylphosphatidylinositol anchor structure is introduced into the DNA.
